(19) United States
(12) Patent Application Publication
(10) Pub. No.: US 2002/0111643 A1
(43) Pub. Date: Aug. 15, 2002

(54) Surgical clip

(75) Inventors: Gebhard Herrmann, Irndorf (DE); Markus Nesper, Tuttlingen (DE); Dieter Weisshaupt, Immendingen (DE)

Correspondence Address: Barry R. Lipsitz, Law Offices of Barry R. Lipsitz, 755 Main Street, Building 8, Monroe, CT, 06468, US

(73) Assignee: AESCULAP AG & Co. KG, Tuttlingen (DE)

(21) Appl. No.: 10/053,245

(22) Filed: Jan. 15, 2002

Related U.S. Application Data
(63) Continuation of application No. PCT/EP00/06126, filed on Jun. 30, 2000.

(30) Foreign Application Priority Data
Jul. 28, 1999 (DE) 199 35 418.9

(51) Int. Cl. A61B 17/08

(52) U.S. Cl. 606/158

(57) ABSTRACT

In order to simplify the structure and production in the case of a surgical clip with two clamping arms, which may be swivelled relative to one another around a rotational axis and in one clamping position have a clamping region where they essentially abut one another, said clamping arms respectively having a free end and an end provided with a bearing, wherein a joint shaft defining the rotational axis is disposed in the two bearings, and with a tension element assigned to the two clamping arms and holding these under prestress in the clamping position, it is proposed that the shaft is formed by the tension element.

The present invention relates to the subject matter disclosed in international application PCT/EP 00/06126 of Jun. 30, 2000, which is incorporated herein by reference in its entirety and for all purposes.

### BACKGROUND OF THE INVENTION

The invention relates to a surgical clip with two clamping arms, which may be swivelled relative to one another around a rotational axis and in one clamping position have a clamping region where they essentially abut one another, said clamping arms respectively having a free end and an end provided with a bearing, wherein a common shaft defining the rotational axis is disposed in the two bearings, and with a tension element associated with the two clamping arms and holding these under prestress in the clamping position, wherein the bearings are supported on the shaft and one of the two bearings comprises a bearing ring through which the shaft passes.

A clip is known according to Heifetz, for example, in which two clip halves are disposed to swivel relative to one another on a bearing pin defining a rotational axis. In addition, a torsion spring surrounding the pin is connected on the inside to the two clip halves in order to hold these under prestress in the clamping position. Such clips are used, for example, to clamp hollow organs, in particular blood vessels.

On this basis, such a clip is composed of at least four structural parts, namely the two clip halves, the torsion spring and the bearing pin. Assembly, amongst other factors, has proved to be disadvantageous, since the bearing pin must be undetachably connected to the two clip halves, so that it cannot fall out unintentionally and be lost, in which case the clip would at the same time separate into its component parts.

A surgical clip of the type described above is known from GB 2 161 206 A1, in which the two clip halves have holes corresponding to the bearing pin so that the two clip halves are supported on the bearing pin serving as shaft.

A further surgical clip is known from DE 89 11 948 U1. It comprises two clamping arms, which may be swivelled relative to one another and which are pressed together by means of a helical spring, the free ends of the helical spring being connected to a respective clip half.

Therefore, the object of the present invention is to configure a clip of the type described above in such a manner as to simplify the structure and production.

### SUMMARY OF THE INVENTION

This object is achieved according to the invention in that the shaft is formed by the tension element.

Accordingly, the tension element has a multiple function in the present invention. It serves as mounting for the two clamping arms and therefore guides and supports them, as a moment generation means in order to hold the clip in its clamping position without the action of additional external forces, and moreover fixes the rotational or swivel axis. In addition, the number of structural parts is reduced from four to three, since a bearing pin serving as shaft is now superfluous. The assembly is thus clearly simplified, since the typically very small bearing pin does not need to be painstakingly connected to the clamping arms. All that is necessary to produce the clip is the defined arrangement of the tension element on the two clamping arms. One clamping arm is supported directly on the shaft via the bearing ring so as to permit rotation of the bearing ring around the shaft as well as an axial displacement of the shaft in the direction of the rotational axis. Accordingly, it is sufficient to secure the shaft relative to the clamping arm in the direction of the rotational axis so that the shaft is not lost.

While the other bearing could also comprise a bearing ring with the shaft passing through it, it is particularly beneficial if the other bearing comprises a bearing shell, which engages only partially around the shaft in circumferential direction. This makes assembly of the two clamping arms particularly simple, for the shaft is inserted through the bearing ring and the bearing shell is placed against the shaft or even mounted thereon, depending on the size of a circumferential angle defined by the bearing shell.

In a preferred embodiment it can be provided that on one of the two clamping arms a counter-bearing is provided, on which the other of the two clamping arms is supported in the direction of the rotational axis. On opening the clip, the force components can act in the direction of the rotational axis determined by the tension element so that there would be a risk of the two clamping arms separating. This is prevented by the counter-bearing, since the forces acting in the direction of the rotational axis are absorbed by the counter-bearing.

It is particularly advantageous in this case if the counter-bearing is formed by a projection disposed on one of the two bearings, the other bearing being supported on said projection on at least one side in the direction of the rotational axis. It is then only necessary to configure a bearing such that it abuts against the projection directly or indirectly so that the counter-bearing can absorb the forces exerted by the one clamping arm via the tension element in the direction of the rotational axis.

A leaf spring, for example, would be conceivable as tension element. However, it is beneficial if the tension element is formed by a helical spring. The elastic forces can be adjusted individually by the thickness of a wire forming the helical spring. Moreover, a helical spring is particularly simple to produce. In addition, the helical spring, which generally has a cylindrical external form, can serve particularly well as shaft for the two bearings.

According to a further preferred embodiment, it can be provided that the tension element has two free ends, which are respectively supported on a clamping arm. In this way it is assured that the torque generated by the tension element is transferred to the clamping arms and holds these together so that the clamping regions of the clamping arms abut one another.

It would be conceivable to undetachably connect the free end of the tension element to one of the two clamping arms. However, it is particularly advantageous if at least one of the free ends of the tension element is angled off and is supported on one of the clamping arms in the area of the clamping region on a side facing away from the clamping region. This results in a detachable connection between clamping arm and tension element without impairing the function and effect of the tension element. The winding of the tension element can be provided at a right angle or also in a U shape, for example.

It is beneficial if another free end of the tension element is angled off and is supported on the other clamping arm in the area of the clamping region on a side facing away from the clamping region. With this configuration, the torque transferred by the tension element in particular on opening of the clip can act, for example, in the transition area between the clamping region and the bearing of the two clamping arms, so that the clamping forces may be adjusted individually by the selection of the tension element and the corresponding selection of such a support area.

However, it may also be advantageously provided that the other free end of the tension element is supported on a tension element abutment disposed on one of the two bearings. By providing the tension element abutment, the abutment surface of the tension element ends against the clamping arms is reduced in the area of the clamping region, as a result of which the number of protruding parts in one placement area of the clip is reduced.

It is beneficial in this case if the tension element abutment is disposed on the bearing ring. The tension element abutment is particularly easy to dispose on the bearing ring, in particular without causing any losses of stability for the bearing ring. The position of the tension element abutment can be arranged as desired over the entire angle range, which permits a variety of configurations.

It is particularly advantageous if the tension element abutment is formed by a recess. A free end of the tension element can be inserted, e.g. by bending it beforehand, into the recess, for example, a drilled hole or notch in or on the bearing ring.

It can be fundamentally provided that each of the two clamping arms has at least one operating element to open the clip. By the action of force on the operating element a torque can be transferred to the clamping arms so that the tension element is subjected to an additional tension force and the clip is opened, i.e. the clamping regions abutting one another in the closed position move away from one another. Depending on the position of the operating elements on the clamping arms, a defined force can be adjusted in this way to open the clip.

In a preferred embodiment of the invention it can be provided that the operating elements are disposed on the bearings. With such an arrangement in proximity to the rotational axis, an increased force must be applied to open the clip, and therefore unintentional opening is not possible even through low forces.

It can be advantageously provided that the operating elements are disposed on the bearings to lie essentially diametrically opposed to the clamping region. In this way, a clip with a particularly narrow configuration can be formed. An elongated structural form results, for example, if the abutting clamping regions run essentially in a straight line. With such a configuration it is similarly not necessary to grip around the clip in order to open it. An application of force on the operating elements is sufficient to cause the clip to open.

It is beneficial if one of the operating elements is disposed in an end region of the bearing shell. Particularly in the case of a bearing shell laterally bordering the bearing ring, the operating element at the same time serves as a connecting element for the two halves of the bearing shell.

In a preferred embodiment of the invention, it can also be provided that the operating element disposed on the other clamping arm is disposed outside a region of the bearing ring bordered by the bearing shell. In this way, the operating element is prevented from penetrating between the bearing shell, so that this can be completely closed on one side, as a result of which the guidance of the bearing ring within the bearing shell is improved. As a result, an additional fixture of the two clamping arms relative to one another can be formed, in particular when a portion of the operating element projects over the bearing shell in the direction of the rotational axis.

The operating elements could be formed by a recess, for example. However, it is particularly advantageous if at least one of the operating elements is formed by an operating projection. Such a projection may be grasped particularly simply, in particular with an applicator tool.

According to a further preferred embodiment of the invention, it can be provided that the operating elements comprise tool receptacles, which have a spherical surface. The applicator tool can be fitted with tool ends corresponding to the tool receptacles which can then constantly fully engage on the tool receptacles relative to one another, i.e. because of the spherical surface and in particular independently of a swivel position of the clamping arms.

Advantageously, it can be provided that at least one of the tool receptacles is formed by an essentially hemispherical recess. A spherical end of an applicator tool can engage particularly readily into such a recess. Moreover, the spherical surface enables the clip to be additionally swivelled, if necessary, around a rotational axis, which runs through the centers of the sphere center points of the spherical ends of the applicator tool defined by the spherical surfaces. This permits individual positioning of the clip in a particularly simple manner.

It is beneficial if at least one of the tool receptacles is formed by an essentially hemispherical projection. In this embodiment, the tool receptacle can be engaged particularly simply by a tool end in the form of a hollow spherical shell, but with all the advantages of the spherical embodiment, in particular of an additional possibility of swivel movement, independently of an opening angle of the clamping arms.

The embodiments described so far would in principle allow an opening angle between the clamping arms to be adjusted to be any desired size. However, it is particularly advantageous if a restriction means is provided on one of the two clamping arms to restrict the opening angle of the clip. In this way, the restoring force of the tension element is restricted at the same time. A possible arrangement would be, for example, to dispose the restriction means on one of the two bearings so that the section of the clamping arm having the clamping region can strike against the restriction means with its side facing away from the clamping region when the clip opens.

According to a preferred embodiment of the invention, it can be fundamentally provided that the two clamping arms cross in a transition area from the clamping region to the bearings. This allows determination of the direction of orientation in which the two bearings must be rotated relative to one another so that the clip opens. Moreover, the clip is protected additionally from falling apart by this engagement of the clamping arms.

The following description of preferred embodiments of the invention is for the purpose of more detailed explanation in association with the drawings.

### BRIEF DESCRIPTION OF THE DRAWINGS

FIG. 1: is a perspective view of a first embodiment of a clip with crossed clamping arms;

FIG. 2: is a perspective view of a second embodiment of a clip with crossed clamping arms;

FIG. 3: is a perspective view of a third embodiment of a clip without crossed clamping arms; and

FIG. 4: is a perspective view of a fourth embodiment of a clip without crossed clamping arms.

### DETAILED DESCRIPTION OF THE INVENTION

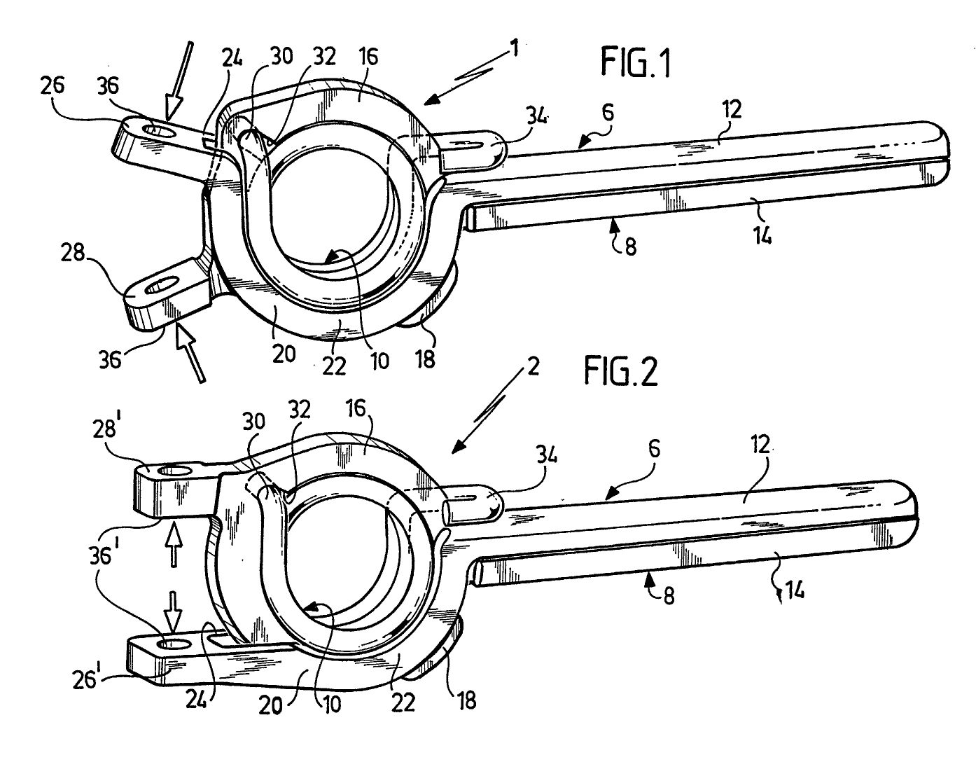

Four clips given the references 1 to 4 overall are shown in FIGS. 1 to 4, each of said clips comprising three structural parts, namely a first clip half 6, a second clip half 8 and a helical spring 10.

The embodiments of clips 1 to 4 described individually below have some elements corresponding to one another, which are provided below with the same reference numerals for reasons of clarity.

Clip 1 has two elongated parallelepipedal clamping arms 12 and 14, which abut against one another essentially over their surface. In an end region of the clamping arm 14 clip half 8 merges into an essentially circular bearing ring 16, the axis of symmetry of which runs transversely to the longitudinal direction of the clamping arm 14. Clamping arm 14 tapers in the transition region so that the bearing ring 16 is slightly thinner than the clamping arm 14 is wide.

At an angle of about 450 relative to the clamping arm 14, a radially projecting counter-bearing 18 extends on the bearing ring 16 in circumferential direction over an angle range of about 250. The counter-bearing 18 constructed as a projection penetrates through a bearing shell 20 engaging on both sides around the bearing ring 16 in the direction of the axis of symmetry thereof, said bearing shell adjoining an end of the clamping arm 12 and its two shell halves 22 and 24 abutting against the bearing ring 16 on both sides. The two shell halves 22 and 24 are connected to one another at their ends facing away from the clamping arm 12 via an operating projection 26. In the case of clip 1, clamping arms 12 and 14 are arranged in a so-called crossed shape. This means that clamping arm 14 on bearing ring 16 is disposed in a region which engages between the two shell halves 22 and 24, which is why it also tapers in one step in the transition area.

A second operating projection 28 crosses the bearing shell 20 in a similar manner by also being disposed in a region on the bearing ring 16 located between the two shell halves 22 and 24. The bearing shell 20 extends over an angle range of about 200°, operating projection 26 and operating projection 28 project radially away from the axis of symmetry of the clamping ring 16 and enclose an angle of about 40°.

Clip 1 is held together by the helical spring 10, the outside diameter of which is essentially adapted to the inside diameter of the bearing ring 16. A free end 30 of the helical spring 10 projects tangentially in the direction of the helix and is finally angled off at 90° and inserted into a recess 32 open towards the axis of symmetry on the bearing ring 16. The other end of the helical spring 10 is firstly angled off at right angles running parallel to clamping arm 14 and engages around the bearing ring 16 with a U-shaped curved portion 34 abutting against clamping arm 12.

When clip 1 is closed, as shown in FIG. 1, the helical spring 10 already stands under a certain prestress, so that the helical spring 10 acting on the bearing ring 16 on one side and on clamping arm 12 on the other side presses the two clamping arms 12 and 14 against one another. The helical spring 10, on the one hand, serves as bearing shaft, on which the bearing ring 16 and the bearing shell 20 are disposed, in which case it defines a rotational axis corresponding to the axis of symmetry of the bearing ring 16 and the helical spring 10.

The doubly crossed clip 1 shown closed in FIG. 1 is opened by the exertion of a mutually opposed force on operating projections 26 and 28 so that operating projections 26 and 28 are moved towards one another to open the clip 1. The side faces of operating projections 26 and 28 facing away from one another are provided with tool receptacles 36 in the form of semispherical recesses. In this way, an applicator tool (not shown) with spherical tool ends can engage into the tool receptacles. The spherical configuration of the tool receptacle 36 and tool end enables an optimum transfer of force in each position of the clip 1. In addition, clip 1 can be swivelled around an axis running through the center points of the spherical tool ends when the tool ends engage into the tool receptacles 36. This enables the clip 1 to be opened with the applicator tool and possibly swivelled to the side when open.

Clip 2 shown in FIG. 2 differs from clip 1 essentially in that only clamping arms 12 and 14 are crossed, as has already been described above. The fastening projections 26′ and 28′ are not crossed, i.e. fastening projection 28′ is disposed on bearing ring 16 in such a way that it does not project between the shell halves 22 and 24 of the bearing shell 20 which extends only over an angle range of about 1500.

When clip 2 is closed, the fastening projections 26′ and 28′ are arranged essentially parallel to one another and tool receptacles 36′ also in the form of hemispherical recesses are disposed in the faces facing one another. To open the clip, the spherical tool ends of the applicator tool must be guided into the tool receptacles 36′ and swivelled away from one another. The direction of operation in the case of clip 2 is exactly the reverse of that of clip 1.

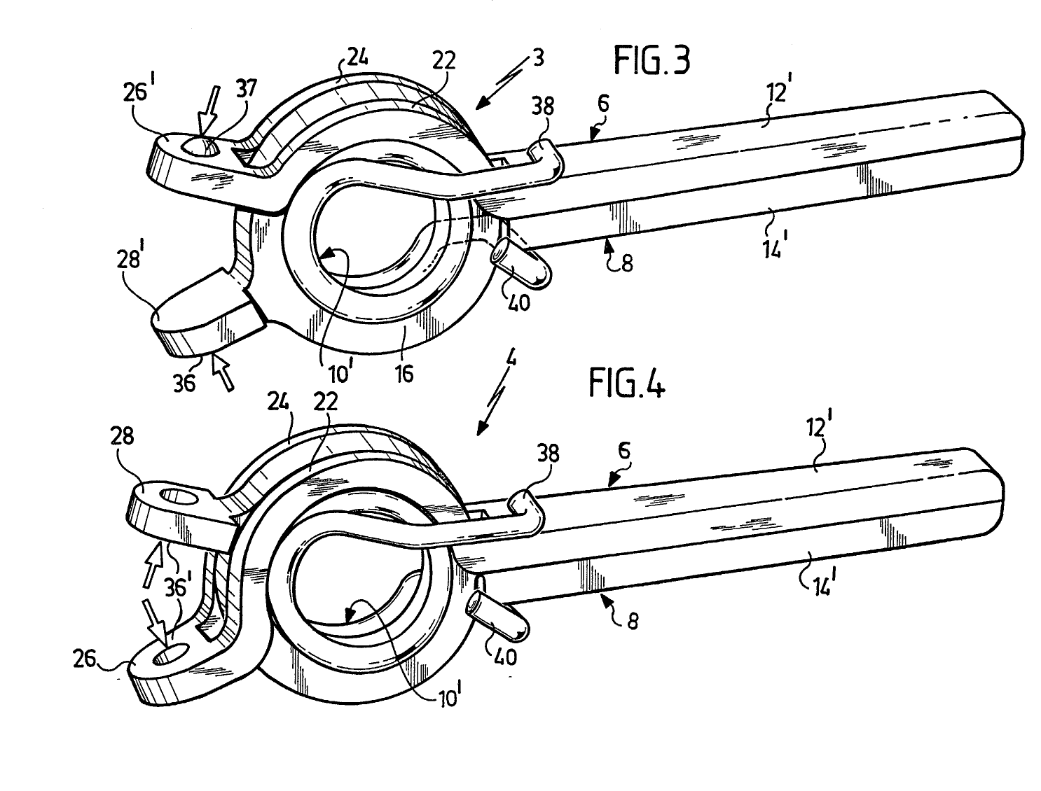

Clip 3 shown in FIG. 3 has non-crossed clamping arms 12′ and 14′ as well as non-crossed operating projections 26′ and 28′. Accordingly, clamping arm 12′ is connected via the two-part bearing shell 20, which comprises shell halves 22 and 24 and extends over an angle range of about 150°, to the operating projection 26′, and clamping arm 14′ is connected via the bearing ring 16 to operating projection 28′, in which case the transition from clamping arm 14′ to bearing ring 16 is not provided between the shell halves 22 and 24, as is the case with clips 1 and 2.

In the case of clip 3 the bearing shaft is also formed by the helical spring 10′, but this has modified ends 38 and 40 compared to the helical spring provided in the case of clips 1 and 2. The helical spring 10′, extending on one side from a last helical winding, projects on an incline and is curved towards the clamping arm 12′, runs essentially parallel to clamping arm 12′ for a short section, is then angled off at right angles and lies on the clamping arm 12′ on its side facing away from clamping arm 14′. The other end of the helical spring 10′ is guided around the bearing ring 16 from the other side in a similar fashion and ends in a U-shaped end 40, in which case a web section extending transversely to two parallel sections of the end 40 abuts against the clamping arm 14′. The end 40 engaging around the bearing ring 16 secures the helical spring 10′ on the clip half 8 and the end 38 secures clip half 6 to clip half 8.

To open the clip 3, the two operating projections 26′ and 28′ enclosing an angle of about 45° must be moved towards one another. On outside faces respectively pointing away from one another, they have hemispherical projections 37 which can engage into a tool end in the form of a hollow sphere of an applicator tool.

Clip 4 shown in FIG. 4 differs from clip 3 in that it has crossed operating projections 26 and 28, such as those also provided in the case of clip 1, i.e. operating projection 28 is arranged on the bearing ring 16 to protrude so that it projects between the shell halves 22 and 24, which extend over an angle range of about 200°. As in the case of clip 2, tool receptacles 36′ formed in a hemispherical shape are disposed on faces of the operating projections 26 and 28 facing one another. To open clip 4, operating projections 26 and 28 must be swivelled away from one another, e.g. by an applicator tool with spherical tool ends engaging into the tool receptacles 36′ and swivelling this.

Alternatively, the tool receptacles 36 or 36′ can also be provided in the form of hemispherical projections 37, as in the case of clip 3, which requires a tool end of the applicator tool accordingly formed in a hemispherical shape. However, in any case a ball connection would be formed between the applicator tool and the tool receptacle.

Application through endoscopic access is possible because of the elongated structural form of clips 1 to 4. It is important to mention that it is not necessary to completely grasp around clips 1 to 4 for opening, all that is necessary is to place the applicator tool on the tool receptacles 36 or 36′.

The direction of movement may be fixed for opening clips 1 to 4 depending on how many crossovers are provided between clamping arms 12 and 14 as well as operating projections 26 and 28. With an even number of crossovers, the clip opens when the operating projections 26 and 28 are moved towards one another, as is the case, for example, with clips 1 and 3, with an odd number of crossovers, the clip opens when the operating projections 26 and 28 are moved away from one another.

What is claimed is:

1. A surgical clip with two clamping arms, which may be swivelled relative to one another around a rotational axis and in one clamping position have a clamping region essentially abutting one another, said clamping arms respectively having a free end and an end provided with a bearing, wherein a common shaft defining the rotational axis is disposed in the two bearings, and with a tension element associated with the two clamping arms and holding these under prestress in the clamping position, wherein the bearings are supported on the shaft and one of the two bearings comprises a bearing ring through which the shaft passes, and wherein the shaft is formed by the tension element.

2. A clip according to claim 1, wherein the other bearing comprises a bearing shell, which engages only partially around the shaft in circumferential direction.

3. A clip according to claim 1, wherein on one of the two clamping arms a counter-bearing is provided, on which the other of the two clamping arms is supported in the direction of the rotational axis.

4. A clip according to claim 3, wherein the counter-bearing is formed by a projection disposed on one of the two bearings, the other bearing being supported on said projection on at least one side in the direction of the rotational axis.

5. A clip according to claim 1, wherein the tension element is formed by a helical spring.

6. A clip according to claim 1, wherein the tension element has two free ends, which are respectively supported on a clamping arm.

7. A clip according to claim 6, wherein at least one of the free ends of the tension element is angled off and is supported on one of the clamping arms in the area of the clamping region on a side facing away from the clamping region.

8. A clip according to claim 6, wherein another free end of the tension element is angled off and is supported on the other clamping arm in the area of the clamping region on a side facing away from the clamping region.

9. A clip according to claim 6, wherein the other free end of the tension element is supported on a tension element abutment disposed on one of the two bearings.

10. A clip according to claim 9, wherein the tension element abutment is disposed on the bearing ring.

11. A clip according to claim 9, wherein the tension element abutment is formed by a recess.

12. A clip according to claim 1, wherein each of the two clamping arms has at least one operating element to open the clip.

13. A clip according to claim 2, wherein each of the two clamping arms has at least one operating element to open the clip.

14. A clip according to claim 13, wherein the operating elements are disposed on the bearings.

15. A clip according to claim 14, wherein the operating elements are disposed on the bearings to lie essentially diametrically opposed to the clamping region.

16. A clip according to claim 14, wherein one of the operating elements is disposed in an end region of the bearing shell.

17. A clip according to claim 15, wherein the operating element disposed on the other clamping arm is disposed outside a region of the bearing ring bordered by the bearing shell.

18. A clip according to claim 16, wherein the operating element disposed on the other clamping arm is disposed in a region of the bearing ring bordered by the bearing shell.

19. A clip according to claim 12, wherein at least one of the operating elements is formed by an operating projection.

20. Clip according to claim 12, wherein the operating elements comprise tool receptacles, which have a spherical surface.

21. A clip according to claim 20, wherein at least one of the tool receptacles is formed by an essentially hemispherical recess.

22. A clip according to claim 20, wherein at least one of the tool receptacles is formed by an essentially hemispherical projection.

23. A clip according to claim 1, wherein the two clamping arms cross in a transition area from the clamping region to the bearings.
